(19) United States
(12) Patent Application Publication
(10) Pub. No.: US 2017/0319408 A1
(43) Pub. Date: Nov. 9, 2017

(54) Menstrual Waste Collection and Disposal Device

(71) Applicant: Jeffrey D. Jackson, Sonora, CA (US)

(72) Inventor: Jeffrey D. Jackson, Sonora, CA (US)

(21) Appl. No.: 15/146,549

(22) Filed: May 4, 2016

(51) Int. Cl. A61F 13/84; A41D 19/00; A61F 13/551

(52) U.S. Cl. CPC A61F 13/84; A61F 13/5518; A61F 13/5515; A41D 19/0075; A61F 2013/8402; A61F 2013/8497

(57) ABSTRACT

A menstrual waste collection and disposal device for sanitary collection and disposal of sanitary napkins, panty liners and tampons includes a shell that has a palm face, a first end and a second end. The first end comprises a first tube, a second tube, and a third tube. The second end is open. A first grip, which is coupled to the first tube, and a second grip, which is coupled to the second tube, are positioned proximate to the first end on the palm face. The shell is configured to receive a hand inserted through the second end positioning a thumb in the first tube, an index finger in the second tube, and the remaining fingers of the hand in the third tube. The first grip and the second grip are configured to facilitate grasping of items and in positioning the items on the palm face of the shell.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIE THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to waste collection and disposal devices and more particularly pertains to a new waste collection and disposal device for sanitary collection and disposal of sanitary napkins, panty liners and tampons.

### BRIEF SUMMARY OF THE INVENTION

An embodiment of the disclosure meets the needs presented above by generally comprising a shell that has a palm face, a first end and a second end. The shell is substantially hand shaped and flexible. The first end comprises a first tube, a second tube, and a third tube. The second end is open. A first grip, which is coupled to the first tube, and a second grip, which is coupled to the second tube, are positioned proximate to the first end on the palm face. The shell is configured to receive a hand inserted through the second end positioning a thumb in the first tube, an index finger in the second tube, and the remaining fingers of the hand in the third tube. The first grip and the second grip are configured to facilitate grasping of items and in positioning the items on the palm face of the shell. The shell is invertible to contain the items positioned on the palm face.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a bottom view of a menstrual waste collection and disposal device according to an embodiment of the disclosure.

FIG. 2 is a top view of an embodiment of the disclosure.

FIG. 3 is an isometric perspective view of an embodiment of the disclosure.

FIG. 4 is a cross-sectional view of an embodiment of the disclosure.

FIG. 5 is an in-use view of an embodiment of the disclosure.

### DETAILED DESCRIPTION OF THE INVENTION

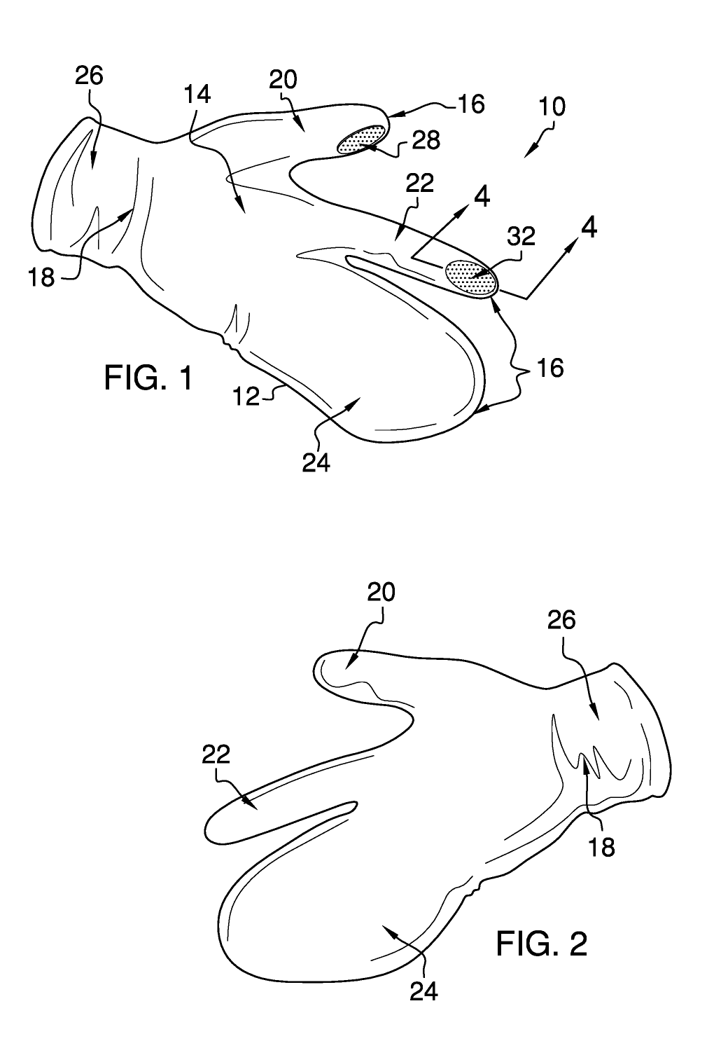

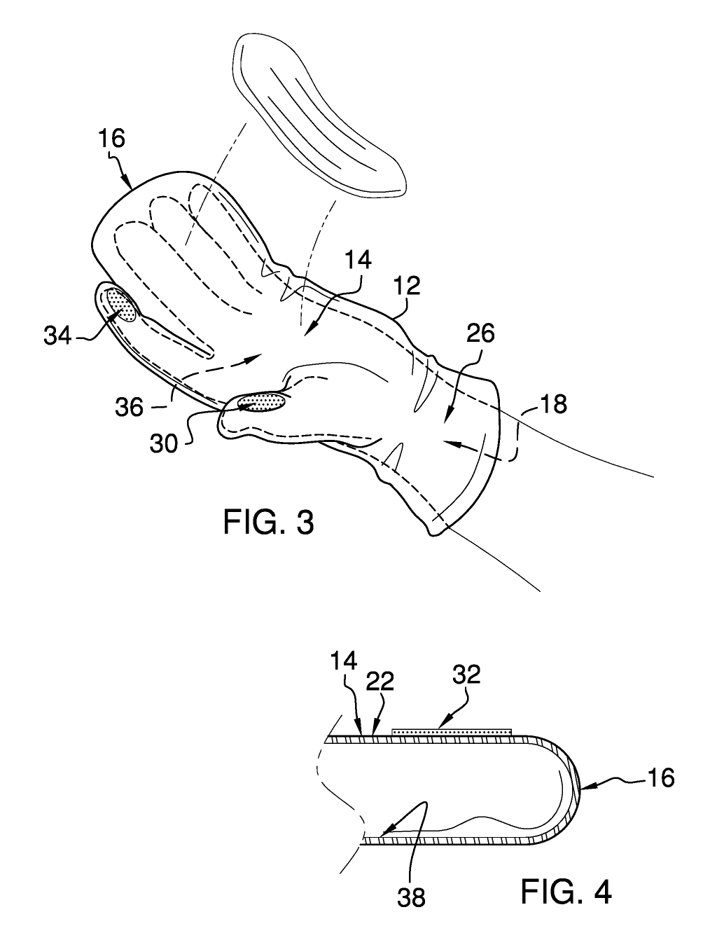

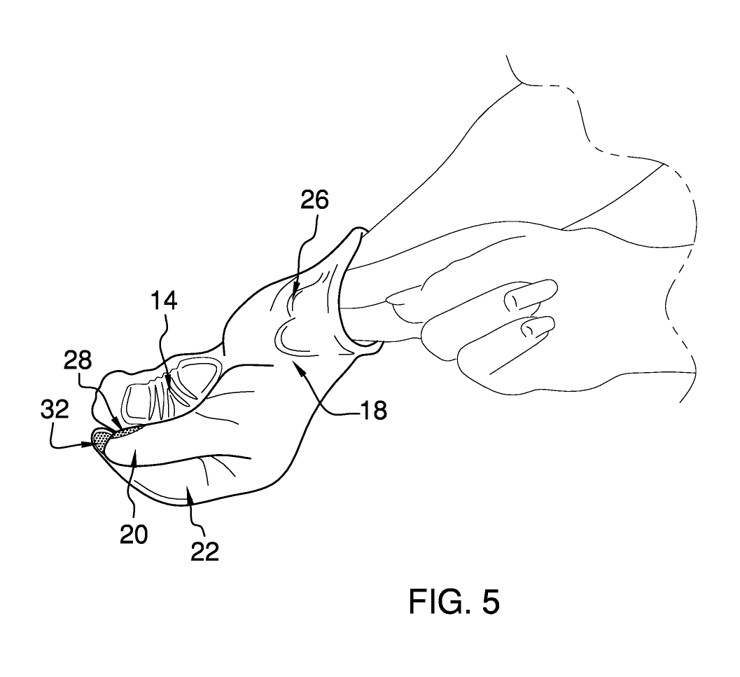

With reference now to the drawings, and in particular to FIGS. 1 through 5 thereof, a new waste collection and disposal device embodying the principles and concepts of an embodiment of the disclosure and generally designated by the reference numeral 10 will be described.

As best illustrated in FIGS. 1 through 5, the menstrual waste collection and disposal device 10 generally comprises a shell 12 that has a palm face 14, a first end 16 and a second end 18. The shell 12 is substantially hand shaped and flexible. The first end 16 comprises a first tube 20, a second tube 22, and a third tube 24. The second end 18 is open. The shell 12 is configured to receive a hand inserted through the second end 18 positioning a thumb in the first tube 20, an index finger in the second tube 22, and the remaining fingers of the hand in the third tube 24.

The shell 12 is impermeable. In one embodiment, the shell 12 comprises latex. In another embodiment, the shell 12 comprises nitrile. In yet another embodiment, the shell 12 comprises rubber.

In one embodiment, the shell 12 is configured for placement on a left hand of a user. In another embodiment, the shell 12 is configured for placement on a right hand of the user.

The shell 12 comprises an extension 26 that is coupled to and extends from the second end 18. The extension 26 is tubular. The extension 26 is positioned on the second end 18 such that the extension 26 is configured to extend over a wrist and at least a portion of a lower arm of the user when the hand is inserted into the shell 12.

A first grip 28 is coupled to the first tube 20 proximate to the first end 16. The first grip 28 is positioned on the palm face 14 of the shell 12. The first grip 28 comprises a plurality of first knobs 30 coupled to and protruding from the first tube 20. A second grip 32 is coupled to the second tube 22 proximate to the first end 16. The second grip 32 is positioned on the palm face 14 of the shell 12. The second grip 32 is positionable adjacent to the first grip 28 when the index finger and the thumb of the hand inserted into the shell 12 are motivated together. The second grip 32 and the first grip 28 engage an object positioned between the second grip 32 and the first grip 28. The second grip 32 comprises a plurality of second knobs 34 coupled to and protruding from the second tube 22.

In one embodiment, indicia 36 are positioned on an interior surface 38 of the shell 12. The indicia 36 are positioned on the interior surface 38 such that the indicia 36 are configured for viewing upon inversion of the shell 12. The indicia 36 indicate proper disposal of the shell 12 and the contents of the shell 12. The indicia 36 comprise lettering, such that the indicia 36 notice against flushing of the shell 12 and the contents of the shell 12.

In use, the shell 12 is configured to receive a hand inserted through the second end 18 positioning a thumb in the first tube 20, an index finger in the second tube 22, and the remaining fingers of the hand in the third tube 24. The first grip 28 and the second grip 32 are configured to facilitate grasping of sanitary napkins, panty liners and tampons. The first grip 28 and the second grip 32 also facilitate positioning of the sanitary napkins, panty liners and tampons on the palm face 14 of the shell 12. The extension 26 is configured for insertion of at least one finger of the user's opposing hand. Inversion of the shell 12 contains the sanitary napkins, panty liners and tampons positioned on the palm face 14 for disposal. The indicia 36 are configured for viewing upon inversion of the shell 12 and indicate proper disposal of the shell 12 and the contents of the shell 12.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A menstrual waste collection and disposal device comprising:
a shell having a palm face, a first end and a second end, said shell being substantially hand shaped, said shell being flexible, said first end comprising a first tube, a second tube, and a third tube, said second end being open, wherein said shell is configured to receive a hand inserted through said second end positioning a thumb in said first tube, an index finger in said second tube, and the remaining fingers of the hand in said third tube;
a first grip coupled to said first tube proximate to said first end, said first grip being positioned on said palm face of said shell;
a second grip coupled to said second tube proximate to said first end, said second grip being positioned on said palm face of said shell, wherein said second grip is positioned on said second tube such that said second grip is positionable adjacent to said first grip when the index finger and the thumb of the hand inserted into said shell are motivated together, wherein said second grip and said first grip engage an object positioned between said second grip and said first grip; and
wherein said shell is configured to receive a hand inserted through said second end positioning a thumb in said first tube, an index finger in said second tube, and the remaining fingers of the hand in said third tube, wherein said first grip and said second grip are configured to facilitate grasping of sanitary napkins, panty liners and tampons and in positioning the sanitary napkins, panty liners and tampons on said palm face of said shell, wherein said second end is configured for insertion of at least one finger of a user's opposing hand, wherein said shell is inverted to contain sanitary napkins, panty liners and tampons positioned on said palm face.

2. The device of claim 1, further including said shell being impermeable.

3. The device of claim 2, further including said shell comprising latex.

4. The device of claim 2, further including said shell comprising nitrile.

5. The device of claim 2, further including said shell comprising rubber.

6. The device of claim 1, further including said shell being configured for placement on a left hand of the user.

7. The device of claim 1, further including said shell being configured for placement on a right hand of the user.

8. The device of claim 1, further including said shell comprising an extension coupled to and extending from said second end, said extension being tubular, wherein said extension is positioned on said second end such that said extension is configured to extend over a wrist and at least a portion of a lower arm of the user when the hand is inserted into said shell.

9. The device of claim 1, further comprising:
said first grip comprising a plurality of first knobs coupled to and protruding from said first tube; and
said second grip comprising a plurality of second knobs coupled to and protruding from said second tube.

10. The device of claim 1, further including indicia positioned on an interior surface of said shell, wherein said indicia are positioned on said interior surface such that said indicia are configured for viewing upon inversion of said shell, wherein said indicia indicate proper disposal of said shell and the contents of said shell.

11. The device of claim 10, further including said indicia comprising lettering, such that said indicia notice against flushing of said shell and the contents of said shell.

12. A menstrual waste collection and disposal device comprising:
a shell having a palm face, a first end and a second end, said shell being substantially hand shaped, said shell being flexible, said first end comprising a first tube, a second tube, and a third tube, said second end being open, wherein said shell is configured to receive a hand inserted through said second end positioning a thumb in said first tube, an index finger in said second tube, and the remaining fingers of the hand in said third tube, said shell being impermeable, said shell comprising latex, said shell being configured for placement on a left hand of a user;
said shell comprising an extension coupled to and extending from said second end, said extension being tubular, wherein said extension is positioned on said second end such that said extension is configured to extend over a wrist and at least a portion of a lower arm of the user when the hand is inserted into said shell;
a first grip coupled to said first tube proximate to said first end, said first grip being positioned on said palm face of said shell, said first grip comprising a plurality of first knobs coupled to and protruding from said first tube;
a second grip coupled to said second tube proximate to said first end, said second grip being positioned on said palm face of said shell, wherein said second grip is positioned on said second tube such that said second grip is positionable adjacent to said first grip when the index finger and the thumb of the hand inserted into said shell are motivated together, wherein said second grip and said first grip engage an object positioned between said second grip and said first grip, said second grip comprising a plurality of second knobs coupled to and protruding from said second tube;
indicia positioned on an interior surface of said shell, wherein said indicia are positioned on said interior surface such that said indicia are configured for viewing upon inversion of said shell, wherein said indicia indicate proper disposal of said shell and the contents of said shell, said indicia comprising lettering, such that said indicia notice against flushing of said shell and the contents of said shell;
wherein said shell is configured to receive a hand inserted through said second end positioning a thumb in said first tube, an index finger in said second tube, and the remaining fingers of the hand in said third tube, wherein said first grip and said second grip are configured to facilitate grasping of sanitary napkins, panty liners and tampons and in positioning the sanitary napkins, panty liners and tampons on said palm face of said shell, wherein said extension is configured for insertion of at least one finger of the user's opposing hand, wherein said shell is inverted to contain sanitary napkins, panty liners and tampons positioned on said palm face, wherein said indicia are configured for viewing upon inversion of said shell and wherein said indicia indicate proper disposal of said shell and the contents of said shell.

13. The device of claim 12, further including said shell being configured for placement on a right hand of the user.

14. The device of claim 12, further including said shell comprising nitrile.

15. The device of claim 12, further including said shell comprising rubber.
